(19) United States
(12) Patent Application Publication
(10) Pub. No.: US 2010/0069893 A1
(43) Pub. Date: Mar. 18, 2010

(54) Technique to enhance voice with Laser beam

(76) Inventor: Kun Yuan Tong, Suwanee, GA (US)

Correspondence Address: Kun Yuan Tong, 2842 Lowe Court, Suwanee, GA, 30024, US

(21) Appl. No.: 12/283,997

(22) Filed: Sep. 17, 2008

(51) Int. Cl. A61B 18/20

(52) U.S. Cl. 606/2; 128/898

(57) ABSTRACT

By tightening string of a musical instrument musician can change the tone and pitch of sound. The more tightening is the sting the stronger is the sound. The more tightening are our vocal cords the high tone and pitch of our voice will be. With the state of art of medical technology laryngologist can burn vocal cords with Laser beam or other heating source to created minimum scars to tighten vocal cords to enhance voice.

### THE BACKGROUND OF THE INVENTION

Some women are suffering from the change of voice after taking hormone pills. Ophthalmologist uses Laser beam to create minimum scars on cornea to flat cornea to correct vision. With the same mechanism laryngologist can burn vocal cords with laser beam to create minimum scars to tighten vocal cords to enhance the tone and pitch of voice. Singer or person who suffers from the change of voice after taking hormone pills can receive Laser surgery to tighten vocal cords improve his/her voice.

### THE BRIEF SUMMARY OF THE INVENTION

Laser beam can burn tissue and create minimum scars. Laser beam can burn cornea to flat cornea to improve vision. Laser beam can burn vocal cords to create minimum scars to change the tone and pitch of voice. Laryngologist can use Laser surgery to improve the performance of singer or to improve the voice of people who has been suffering from the change of voice after taking hormone pills. With the state of art of medical technology laryngologist can create a singer.

### THE DRAWINGS OF THE INVENTION

FIG. 1 is perspective view of vocal cords.

FIG. 2 is perspective view of interrupted longitudinal lines on vocal cords that have been burnt and created by Laser beam.

FIG. 3 is perspective view of continuous longitudinal lines on vocal cords that have been burnt and created by Laser beam.

FIG. 4 is perspective view of interrupted transverse lines on vocal cords that have been burnt and created by Laser beam.

FIG. 5 is perspective view of continuous transverse lines on vocal cords that have been burnt and created by Laser beam.

### THE DETAILED DESCRIPTION OF THE INVENTION

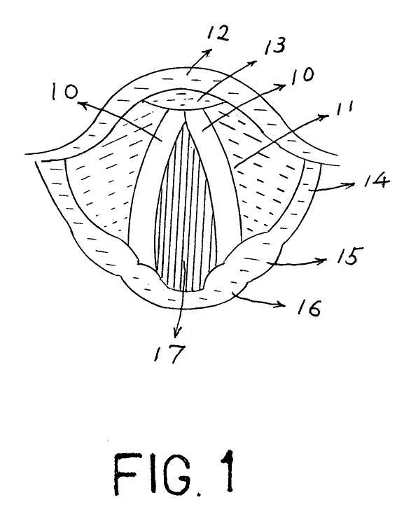

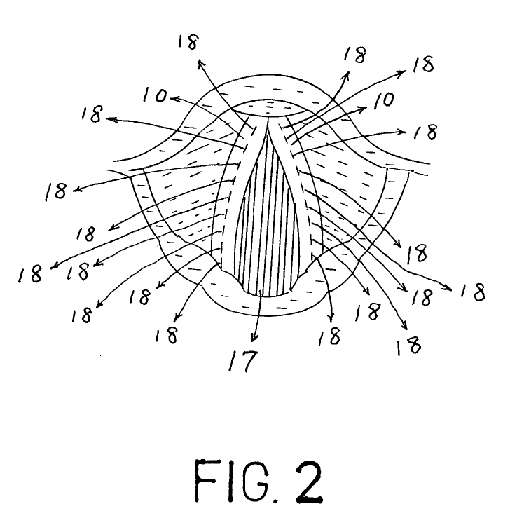

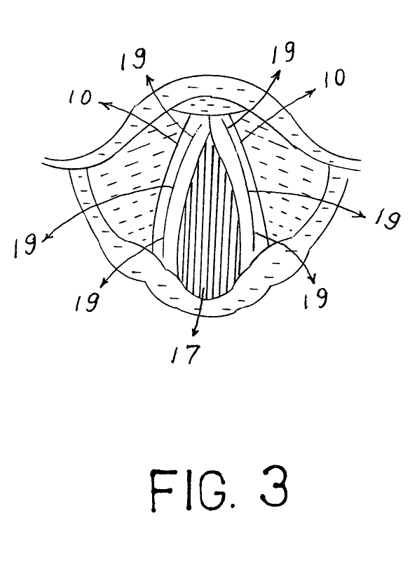

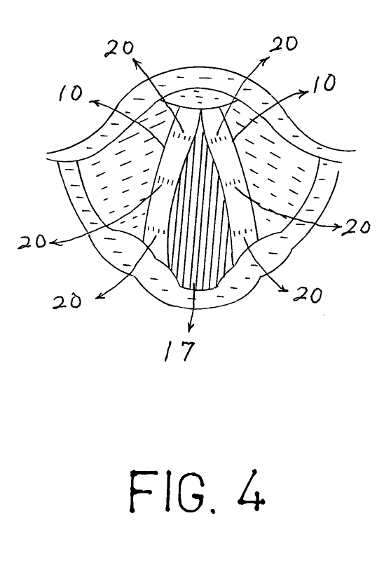

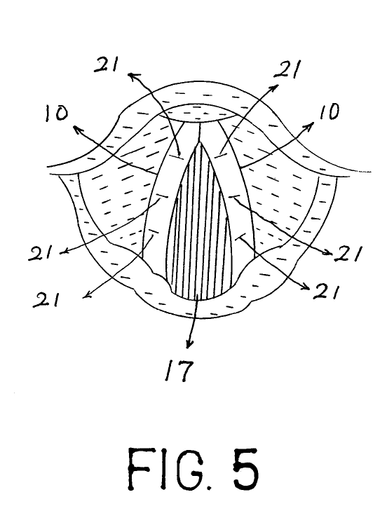

Referring now in the detail to the drawings numeral 10 of FIG. 1 is vocal cords. Numeral 11 of FIG. 2 is interrupted longitudinal lines that have been burnt and created by Laser beam. Numeral 12 of FIG. 3 is continuous longitudinal lines that have been burnt and created by Laser beam. Numeral 13 of FIG. 4 is interrupted transverse lines that have been burnt and created by Laser beam. Numeral 14 of FIG. 5 is continuous transverse lines that have been burnt and created by Laser beam. The interrupted longitudinal lines 11, the continuous longitudinal lines 12, the interrupted transverse lines 13 and the continuous transverse lines 14 that were burnt and created by Laser beam will turn into minimum scars to tighten vocal cords to enhance voice. Other heating source that burns and creates minimum scars to tissue can substitute Laser beam to do the same job.

1. To create minimum scars on vocal cords created by means of burning with Laser beam or other heating source.

2. To create minimum scars on vocal cords in claim 1 is to create multiple interrupted longitudinal scars on vocal cords.

3. To create minimum scars on vocal cords in claim 1 is to create multiple continuous longitudinal scars on vocal cords.

4. To create minimum scars on vocal cords in claim 1 is to create multiple interrupted transverse scars on vocal cords.

5. To create minimum scars on vocal cords in claim 1 is to create multiple continuous transverse scars on vocal cords.
